(19) United States
(12) Patent Application Publication
(10) Pub. No.: US 2021/0169373 A1
(43) Pub. Date: Jun. 10, 2021

(54) GAIT PROFILER SYSTEM AND METHOD

(71) Applicant: B-TEMIA INC., St-Augustin-de-Desmaures (CA)

(72) Inventors: Nathaniel ZOSO, Québec (CA); Victorien THIAUX, Québec (CA); Kevin BOUCHARD, Québec (CA); Katia BILODEAU, Québec (CA); Hugues LAVOIE, St-Augustin-de-Desmaures (CA)

(21) Appl. No.: 16/072,869

(22) PCT Filed: Jan. 25, 2017
(86) PCT No.: PCT/CA2017/000016

§ 371 Date: Jul. 25, 2018

Related U.S. Application Data
(60) Provisional application No. 62/286,902, filed on Jan. 25, 2016.

(51) Int. Cl. A61B 5/11; A61B 5/00

(52) U.S. Cl. CPC A61B 5/112; A61B 5/1126; A61B 5/1118; A61H 3/00; A61B 5/6807; A61B 2562/0219; A61B 5/6812

(57) ABSTRACT

A system and method for determining the gait profile of a user. The gait profiler system uses sensing systems that include inertial sensors configured to be positioned at the right and left foot-ankle structure, as well as spatial orientation of lower extremity body segments (shanks, thighs, and trunk) of the person for which the gait profile is to be determined. In an illustrative embodiment, the gait profiler system uses two additional inertial sensors at the left and right leg-knee or thigh-hip structure as well as sensors providing information indicative of the angular positions of the left and right knee and thigh, which may be provided by an exoskeleton or orthotic devices worn by the user. The determination of the gait profile of the user is then performed using biomechanics information about the user from the inertial sensors combined with the knee and hip angles.

### CROSS-REFERENCE TO RELATED APPLICATIONS

This application claims the benefits of U.S. provisional patent application No. 62/286,902 filed on Jan. 25, 2016, which is herein incorporated by reference.

### TECHNICAL FIELD

The present disclosure relates to a gait profiler system and method for determining the gait profile of a user.

### BACKGROUND

Assistive mobility device, such as actuated orthoses, providing optimal knee assistance (i.e. energy injection during more than 99% of the user's activities) require knowledge of the state of the leg of the user, that is either a) in a stance state (i.e. in contact with the ground or b) in a swing state.

A common method of accomplishing this is using pressure sensors. However, this method has drawbacks, mainly:
pressure sensors have to be placed into the sole of the user's shoe or create a sole that can be adapted to fit all shoes;
the accuracy of the pressure sensors is sensitive to the user's specific stance, (depends on which part of the foot is pressed when engaged to the ground), unless a plurality of pressure sensors are used; and
pressure sensors can be brittle and break after a short use.

Accordingly, there is a need for a gait profiler system and method for determining the gait profile of a user that overcomes the pressure sensor's drawbacks.

### SUMMARY

The present disclosure provides a gait profiler system for determining the gait profile of a user, comprising:
a first sensing system associated with a right foot of the user, including:
a first inertial sensor;
a first securing mechanism configured to secure the first sensing system to the right foot of the user;
a first set of external sensors observing a right shank, thigh and trunk spatial orientation;
a second sensing system associated with a left foot of the user, including:
a second inertial sensor;
a second securing mechanism configured to secure the second sensing system to the left foot of the user;
a second set of external sensors observing a left shank, thigh and trunk spatial orientation;
at least one processor in communication with the first and second inertial sensors and the first and second sets of external sensors, the at least one processor having an associated memory comprising instructions stored thereon, that when executed on the processor perform the steps of:
receiving biomechanics information about the user from the first and second inertial sensors;
receiving biomechanics information from the first and second sets of external sensors;
generating locomotion-related information for the right foot of the user using the biomechanics information from the first inertial sensor and the first set of external sensors;
generating locomotion-related information for the left foot of the user using the biomechanics information from the second inertial sensor and the second set of external sensors;
calculating a locomotion state of the user (for example stance state or swing state) by merging the locomotion-related information of the right foot and left foot; and
generating the gait profile of the user using the locomotion state of the user.

The present disclosure also provides a gait profiler system as described above, wherein the first and second sets of external sensors include a pair of inertial sensors configured to be positioned at respective right and left leg-knee or thigh-hip structures and a plurality of sensors providing information indicative of the angular positions of the right and left knee and thigh of the user.

The present disclosure further provides a gait profiler system as described above, wherein the various sensors are provided by an exoskeleton or orthotic devices worn by the user.

The present disclosure further provides a gait profiler system as described above, wherein the step of merging the locomotion-related information of the right foot and of the left foot of the user is performed using a sensor fusion algorithm comprising the sub-steps of:
determining a static state of each of the right foot and of the left foot of the user using the locomotion-related information of the right foot and of the left foot;
determining a dynamic state of each of the right foot and of the left foot of the user using the locomotion-related information of the right foot and of the left foot; and
determining the locomotion state of the user using the static state and the dynamic state of each of the right foot and of the left foot of the user.

The present disclosure still further provides a gait profiler system as described above, wherein the step of generating the gait profile of the user includes the sub-steps of:
calculating secondary gait information using at least one of the biomechanics information about the user from the first and second inertial sensors and the biomechanics information from the first and second sets of external sensors;
calculating the gait profile based on the locomotion state of the user, the locomotion state having an associated model gait profile; and
optimizing the gait profile based on the secondary gait information.

### BRIEF DESCRIPTION OF THE FIGURES

Embodiments of the disclosure will be described by way of examples only with reference to the accompanying drawings, in which:

FIG. 1 is a schematic representation of a gait profiler system;

FIG. 2 is a schematic representation of the gait profiler system in accordance with an illustrative embodiment of the present disclosure;

FIG. 3 is a flow diagram of the state calculation process in accordance with the illustrative embodiment of the present disclosure;

FIG. 4 is a flow diagram of the sensor fusion algorithm sub-steps of the state calculation process of FIG. 3; and

FIG. 5 is a flow diagram of the gait profile calculation process in accordance with the illustrative embodiment of the present disclosure.

Similar references used in different Figures denote similar components.

### DETAILED DESCRIPTION

Generally stated, the non-limitative illustrative embodiment of the present disclosure provides a system and method for determining the gait profile of a user. The gait profiler system uses sensing systems that include inertial sensors configured to be positioned at the right and left foot-ankle structure, as well as spatial orientation of lower extremity body segments (shanks, thighs, and trunk) of the person for which the gait profile is to be determined. In an illustrative embodiment, the gait profiler system uses two additional inertial sensors at the left and right leg-knee or thigh-hip structure as well as sensors providing information indicative of the angular positions of the left and right knee and thigh, which may be provided by an exoskeleton or orthotic devices worn by the user, such as described, for example, in U.S. Pat. No. 9,370,439 entitled “LOAD DISTRIBUTION DEVICE FOR HUMAN JOINTS”. This determination of the gait profile of the user is performed using biomechanics information about the user from the inertial sensors combined with the knee and hip angles.

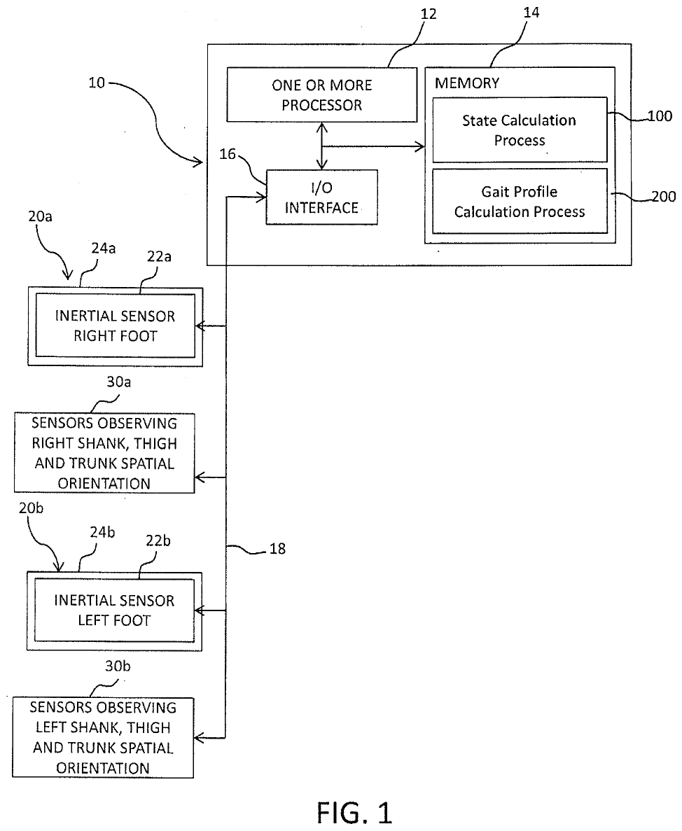

Referring to FIG. 1, the gait profiler system 10 includes one or more processor 12 with an associated memory 14 comprising instructions stored thereon, that when executed on the processor 12, perform the steps of the state calculation process 100 and the gait profile calculation process 200, which processes will be further described below, and an input/output (I/O) interface 16 for communication with a right foot 20a and a left foot 20b sensing systems and external sensors observing the right 30a and left 30b shank, thigh and trunk spatial orientation through communication link 18, which may be wired, wireless or a combination of both.

Each of the sensing systems 20a, 20b includes, respectively, an associated inertial sensor 22a, 22b (providing biomechanics information about a respective foot of the user) and a securing mechanism 24a, 24b configured to secure the sensing systems 20a, 20b, for example, right below the medial malleolus of an associated foot of the user.

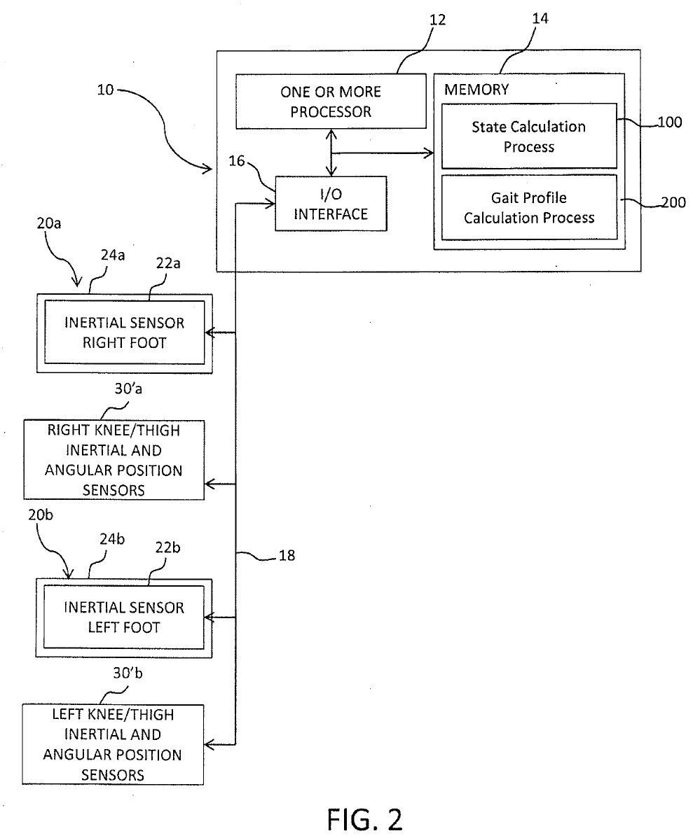

In an illustrative embodiment of the gait profiler system 10, shown in FIG. 2, the external sensors 30a, 30b take the form of right 30′a and left 30′b knee or thigh inertial and knee and hip angular positions sensors.

It is to be understood that the knee and hip angular position sensors 30′a, 30′b may take the form of any sensors providing information indicative of angular position or from which angular position may be generated as the knee and hip angles may be determine by direct measurement or deduced from biomechanics information provided by a variety of types of sensors.

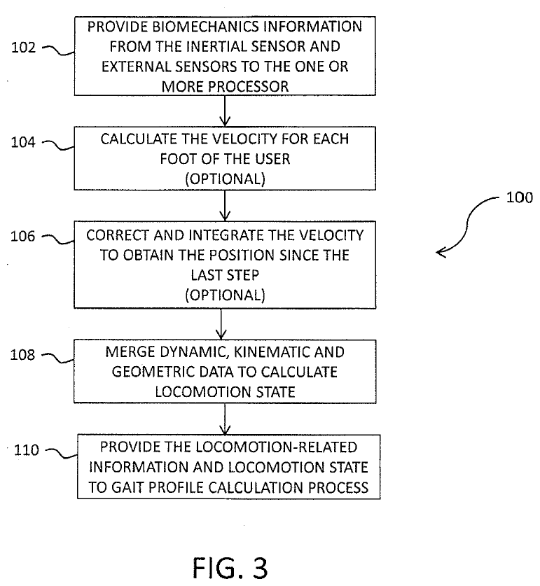

Referring to FIG. 3, there is shown a flow diagram of the state calculation process 100 executed by the one or more processor 12 (see FIGS. 1 and 2) in accordance with the illustrative embodiment of the present disclosure. Steps of the process 100 are indicated by blocks 102 to 110.

The process 100 starts at block 102 where the biomechanics information and knee and hip angles from the associated inertial sensors 20a, 20b and the external sensors 30a, 30b are provided to the one or more processor 12.

At block 104, optionally, the velocity is calculated by integrating the acceleration expressed in the global coordinates system.

At block 106, optionally, the velocity is corrected and integrated to obtain the position since the last step.

Then, at block 108, the position and velocity (if optional steps 104 and 106 are performed), acceleration, rotation and orientation are merged with a sensor fusion algorithm in order to calculate the locomotion state (i.e. stance or swing state) of the user.

Finally, at block 110, the process 100 provides the stance or swing state of the user and the the locomotion-related information of each foot of the user to the gait profile calculation process 200.

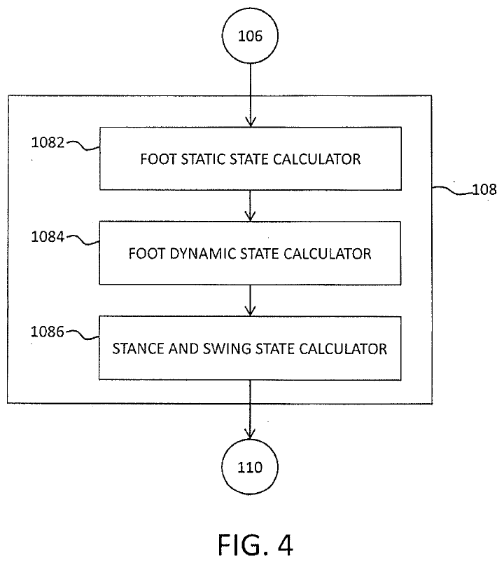

Referring to FIG. 4, there is shown a flow diagram of the sensor fusion algorithm sub-steps used in step 108 of the state calculation process 100 of FIG. 3. The sensor fusion algorithm sub-steps are indicated by blocks 1082 to 1086.

At block 1082, the static state of each of the right foot and left foot of the user is determined using the biomechanics information, i.e. is the foot in contact with the ground and is motionless or not, etc.

Then, at block 1084, the dynamic state of each of the right and left foot of the user is determined using the biomechanics information, i.e. is the foot in motion, is it part of a locomotion cycle or not, etc.

Finally, at block 1086, the algorithm determines the locomotion state (i.e. stance or swing state) of the user. To this end, the static and dynamic states of the right foot and the left foot are used (i.e. static right foot, static left foot, dynamic right foot, dynamic left foot), the various combinations of the right foot and left foot states determining if the user is in a stance or swing state. It is to be understood that other biomechanics information may be used to complement the static and dynamic states of the right foot and the left foot.

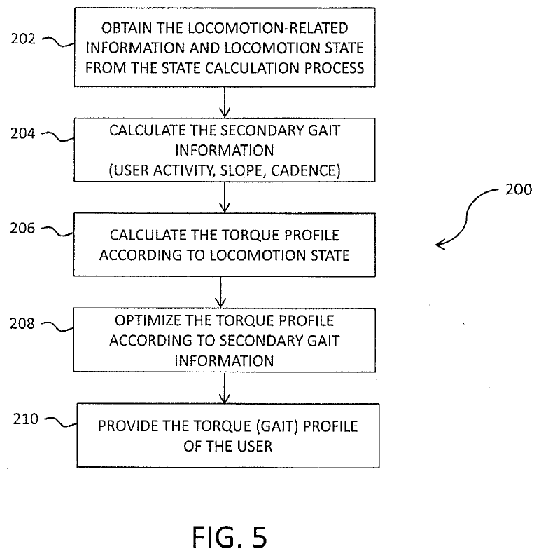

Referring now to FIG. 5, there is shown a flow diagram of the gait profile calculation process 200 executed by the one or more processor 12 (see FIGS. 1 and 2) in accordance with the illustrative embodiment of the present disclosure. Steps of the process 200 are indicated by blocks 202 to 210.

The process 200 starts at block 202 where the locomotion state (i.e. stance or swing state) of the user and the the locomotion-related information of each foot of the user is obtained from the state calculation process 100 (see FIG. 3).

At block 204, the secondary gait information such as user activity, slope, cadence, etc., is calculated from the biomechanics information and knee and hip angles.

At block 206, a torque profile is calculated based on the stance or swing state of the user. Each state is provided with a model torque profile, i.e. stance state torque and swing state torque profiles

Then, at block 208, the torque profile is optimized based on the user secondary gait information. This means that when a change of locomotion state and/or secondary gait information is detected, the torque profile is adjusted in order to limit the effects of those changes on the gait of the user.

Finally, at block 210, the process 200 provides the torque profile (i.e. gait profile) of the user.

It is to be understood that in alternative embodiments the state calculation process 100 and the gait profile calculation process 200 may be executed on a single or separate processors 12 and that the state calculation process 100 may be executed on separate processors 12 for the right and left foot of the user, the inertial sensors 20a, 20b and external sensors 30a, 30b providing their information directly to their associated processor 12.

Although the present disclosure has been described by way of particular non-limiting illustrative embodiments and examples thereof, it should be noted that it will be apparent to persons skilled in the art that modifications may be applied to the present particular embodiment without departing from the scope of the present disclosure as hereinafter claimed.

1. A gait profiler system for determining the gait profile of a user, comprising:
a first sensing system associated with a right foot of the user, including:
a first inertial sensor;
a first securing mechanism configured to secure the first sensing system to the right foot of the user;
a first set of external sensors observing a right shank, thigh and trunk spatial orientation;
a second sensing system associated with a left foot of the user, including:
a second inertial sensor;
a second securing mechanism configured to secure the second sensing system to the left foot of the user;
a second set of external sensors observing a left shank, thigh and trunk spatial orientation;
at least one processor in communication with the first and second inertial sensors and the first and second sets of external sensors, the at least one processor having an associated memory comprising instructions stored thereon, that when executed on the processor perform the steps of:
receiving biomechanics information about the user from the first and second inertial sensors;
receiving biomechanics information from the first and second sets of external sensors;
generating locomotion-related information for the right foot of the user using the biomechanics information from the first inertial sensor and the first set of external sensors;
generating locomotion-related information for the left foot of the user using the biomechanics information from the second inertial sensor and the second set of external sensors;
calculating a locomotion state of the user by merging the locomotion-related information of the right foot and of the left foot; and
generating the gait profile of the user using the locomotion state of the user.

2. The gait profiler system of claim 1, wherein the first and second sets of external sensors include a pair of inertial sensors configured to be positioned at respective right and left leg-knee or thigh-hip structures and a plurality of sensors providing information indicative of the angular positions of the right and left knee and thigh of the user.

3. The gait profiler system of claim 2, wherein the inertial sensors at the left and right leg-knee or thigh-hip structures and the plurality of sensors providing information indicative of the angular positions of the right and left knee are provided by an exoskeleton or orthotic devices worn by the user.

4. The gait profiler system of claim 1, wherein the biomechanics information provided by the inertial sensors include acceleration and the steps of generating locomotion-related information for the right foot and the left foot of the user include calculating a velocity by integrating the acceleration expressed in the a global coordinates system.

5. The gait profiler system of claim 4, wherein the steps of generating locomotion-related information for the right foot and the left foot of the user include calculating a position by integrating the velocity.

6. The gait profiler system of claim 5, wherein the step of merging the locomotion-related information of the right foot and left foot of the user includes merging the velocity and the position.

7. The gait profiler system of claim 1, wherein the step of generating the gait profile of the user further uses the locomotion-related information of the right foot and of the left foot.

8. The gait profiler system of claim 1, wherein the step of merging the locomotion-related information of the right foot and of the left foot of the user is performed using a sensor fusion algorithm.

9. The gait profiler system of claim 8, wherein the sensor fusion algorithm comprises the sub-steps of:
determining a static state of each of the right foot and of the left foot of the user using the locomotion-related information of the right foot and of the left foot;
determining a dynamic state of each of the right foot and of the left foot of the user using the locomotion-related information of the right foot and of the left foot; and
determining the locomotion state of the user using the static state and the dynamic state of each of the right foot and the left foot of the user.

10. The gait profiler system of claim 9, wherein the sub-step of determining the locomotion state of the user further uses the locomotion-related information for the right foot and the left foot of the user.

11. The gait profiler system of claim 1, wherein the step of generating the gait profile of the user includes the sub-steps of:
calculating secondary gait information using at least one of the biomechanics information about the user from the first and second inertial sensors and the biomechanics information from the first and second sets of external sensors;
calculating a gait profile based on the locomotion state of the user, the locomotion state having an associated model gait profile; and
optimizing the gait profile based on the secondary gait information.

12. The gait profiler system of claim 11, wherein the secondary gait information includes at least one of a user activity, a slope and a cadence.

13. The gait profiler system of claim 1, wherein the locomotion state of the user is one of a stance state and a swing state.
